(19) United States
(12) Patent Application Publication
(10) Pub. No.: US 2016/0286860 A1
(43) Pub. Date: Oct. 6, 2016

(54) ELECTRONIC VAPORIZING DEVICE HAVING AN AUTOMATICALLY RE-SEALABLE RESERVOIR TANK

(71) Applicant: Richard Reed FLAYLER, Livermore, CA (US)

(72) Inventor: Richard Reed FLAYLER, Livermore, CA (US)

(21) Appl. No.: 14/673,874

(22) Filed: Mar. 31, 2015

(51) Int. Cl. A24F 47/00; A61M 11/04

(52) U.S. Cl. CPC A24F 47/008; A61M 11/04

(57) ABSTRACT

An electronic vaporizing device has a reservoir tank that reseals after penetration. A reservoir tank sealed with the septum membrane is exposed for refilling (and initially filling) a liquid solution with a syringe needle. The liquid solution contains an active ingredient such as nicotine, THC or pharmaceutical. The septum membrane is be pierced by the syringe needle and then automatically resealed when withdrawn due to durometer and anti-coring characteristics of the septum membrane.

### FIELD OF THE INVENTION

The invention relates generally to electronic vaporizing devices, and more specifically, to a reservoir tank that reseals after penetration by a syringe needle or other means such as automated or manual dispensing equipment that fills the reservoir tank with a liquid solution.

### BACKGROUND

Vaporizers are quickly gaining market share from conventional combustible products as a safer alternative. For example, vaporizers for use with tobacco and marijuana allow consumption of active ingredients without the carcinogens associated with smoking the same. Vaporizers are more convenient than smoking products because they can be activated for just one or two inhalations while traditional paper and leaf products are typically single use, or create a lot of waste during reuse. Also, vaporizers are more durable from plastic and rubber construction, allowing them to withstand significantly more weight.

Disposable vaporizers come pre-packaged with a liquid solution containing an active ingredient. Once consumed, the entire vaporizer is thrown away. Refillable vaporizers typically are of higher quality than disposables, and the liquid solution can be refilled. Generally, the vaporizer is formed from many pieces, some of which are detached or unscrewed to reach a reservoir holding the liquid solution. The reservoir is removed and opened, or even opened in situ, for access to refill (or initially fill). Afterwards, the reservoir is closed and re-secured.

However, the process of manually filling liquid solution can be inconvenient and complicated due to spillage when trying to direct the liquid solution into a small chamber. Conventional manufacturing processes can require strict timing in order to prevent leakage. In more detail, reservoir tanks that hold solutions have holes on the bottom for use during vaporizing, so if reservoir tanks are not quickly plugged to create a vacuum, the leakage can be problematic. Some liquid solutions are oil-based and can be difficult to clean.

It would be desirable to address these and other shortcomings of electronic vaporizing devices with an improved mechanism for filling up or refilling a reservoir with liquid solution.

### SUMMARY

The above shortcomings are addressed by an electronic vaporizing device having a reservoir tank that reseals after penetration by a syringe needle that fills the reservoir tank with a liquid solution, by methods operating therein, and by methods of manufacture.

In one embodiment, a reservoir tank of an electronic vaporizing device is sealed with a septum membrane prior to filling with a liquid solution. The septum membrane can be pierced by a syringe needle through a mouth piece (or butt cap) and then automatically reseal when the syringe needle is withdrawn in order to contain the liquid solution. The liquid solution can contain an active ingredient such nicotine, THC (tetrahydrocannabinol), or pharmaceuticals suspended in oil, alcohol or other solvents. The same mouth piece is used to inhale the liquid solution with a vacuum force when vaporized by a battery-powered atomizer.

In one implementation, the septum membrane is supported by a PFTE (polytetrafluoroethylene) backing and secured in place against the reservoir tank with a reservoir cap.

In another implementation, the syringe needle includes a hole to relieve air or other gas displaced by the injected liquid solution.

Advantageously, electronic vaporizing devices can be filled (and refilled) in an easier manner, in both manual and manufacturing environments, for the convenient administration of nicotine, THC, pharmaceuticals or therapeutics. Further, manufacturing process defects are eliminated and productivity is increased with the immediate sealing of the septum membrane.

### BRIEF DESCRIPTION OF THE DRAWINGS

In the following drawings, like reference numbers are used to refer to like elements. The Figures depict various embodiments for purposes of illustration only. One skilled in the art will readily recognize from the following discussion that other embodiments of the structures and methods illustrated herein may be employed without departing from the described principles.

FIG. 1 is a perspective diagram illustrating a detailed exploded view of a re-sealable septum assembly within an exploded view of an electronic vaporizing device, according to one embodiment of the present invention.

FIG. 2 is a perspective diagram illustrating a connection between a main butt assembly and a battery assembly, according to some embodiments of the present invention.

FIG. 3 is a perspective diagram illustrating an exploded view of the main butt assembly, according to one embodiment.

FIGS. 4A-B are perspective diagrams and machine drawings showing further details of the reservoir tank assembly, according to one embodiment.

FIGS. 5A-B are perspective diagrams and machine drawings showing further details of the septum assembly, according to one embodiment.

FIG. 6 is a flow chart illustrating a method for filling a re-sealable reservoir tank with an electronic vaporizing device, according to one embodiment.

### DETAILED DESCRIPTION

The present disclosure provides an electronic vaporizing device having a reservoir tank that reseals after penetration by a syringe needle which fills the reservoir tank with a liquid solution, methods operating therein, and methods of manufacture.

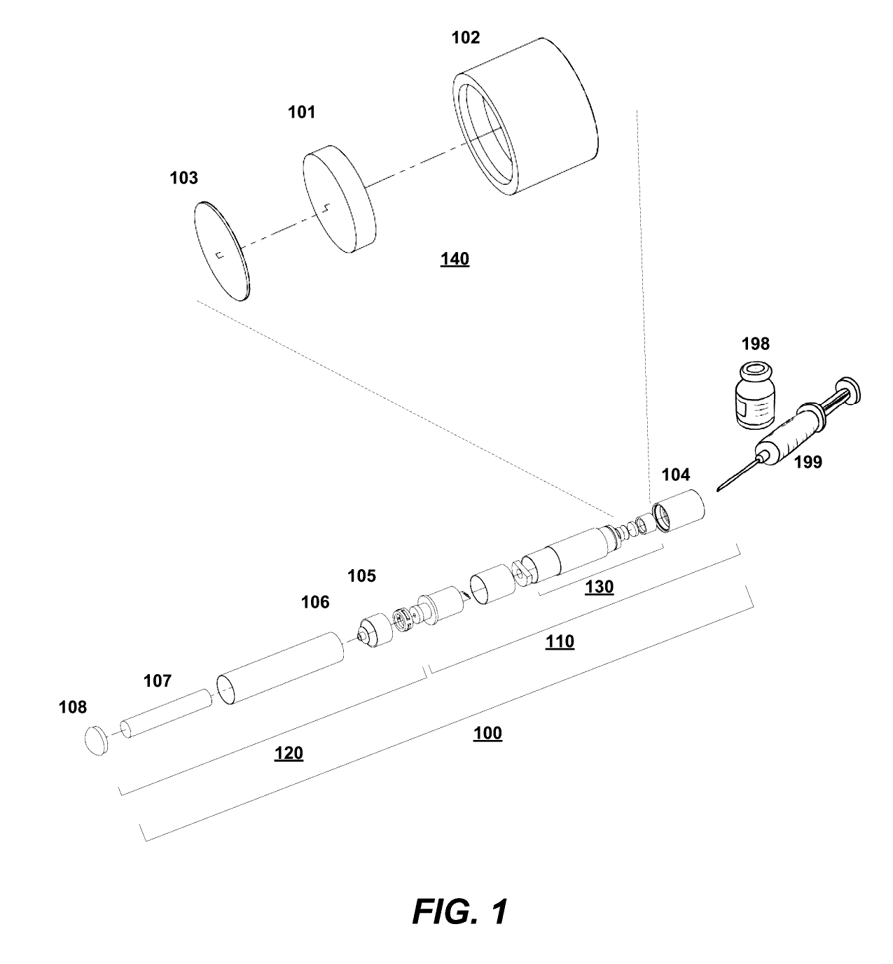

FIG. 1 is a perspective diagram illustrating a detailed exploded view of a re-sealable septum assembly 140 within an exploded view of an electronic vaporizing device 100, according to one embodiment of the present invention. The electronic vaporizing device 100 generally comprises a main butt assembly 110 and a battery tube assembly 120. The components are manufactured using primarily plastics and rubbers, and can be formed from one pour or be manufactured separately. The electronic vaporizing device 100 can be disposable or refillable, and is suitable for dispersion of tobacco vapor, marijuana vapor, other pharmaceutical or therapeutic vapor, and the like. A liquid solution such as propylene glycerol or other carrier oil or carrier agent that vaporizes includes an extract or active ingredient. The electronic vaporizing device 100 can be shaped like a cigarette, and generally have a small or a large form factor.

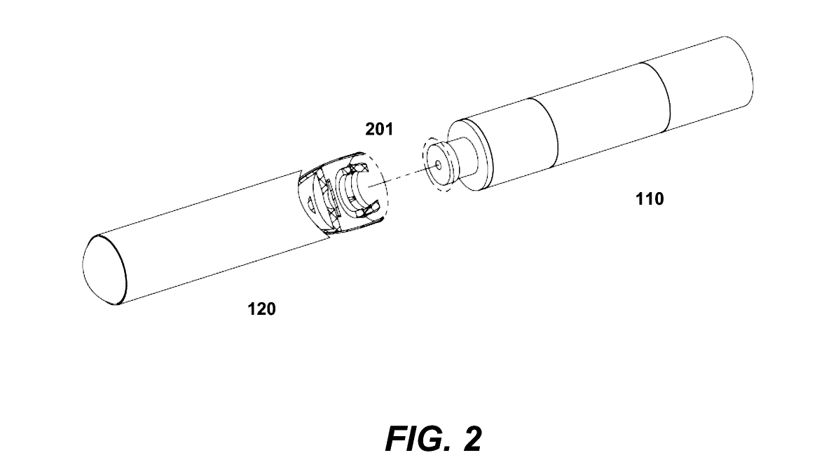

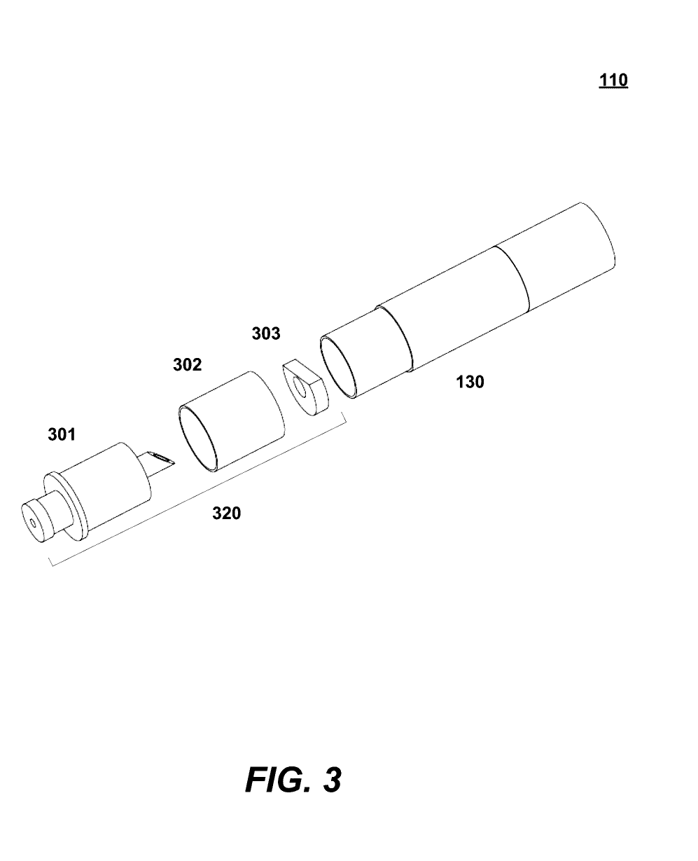

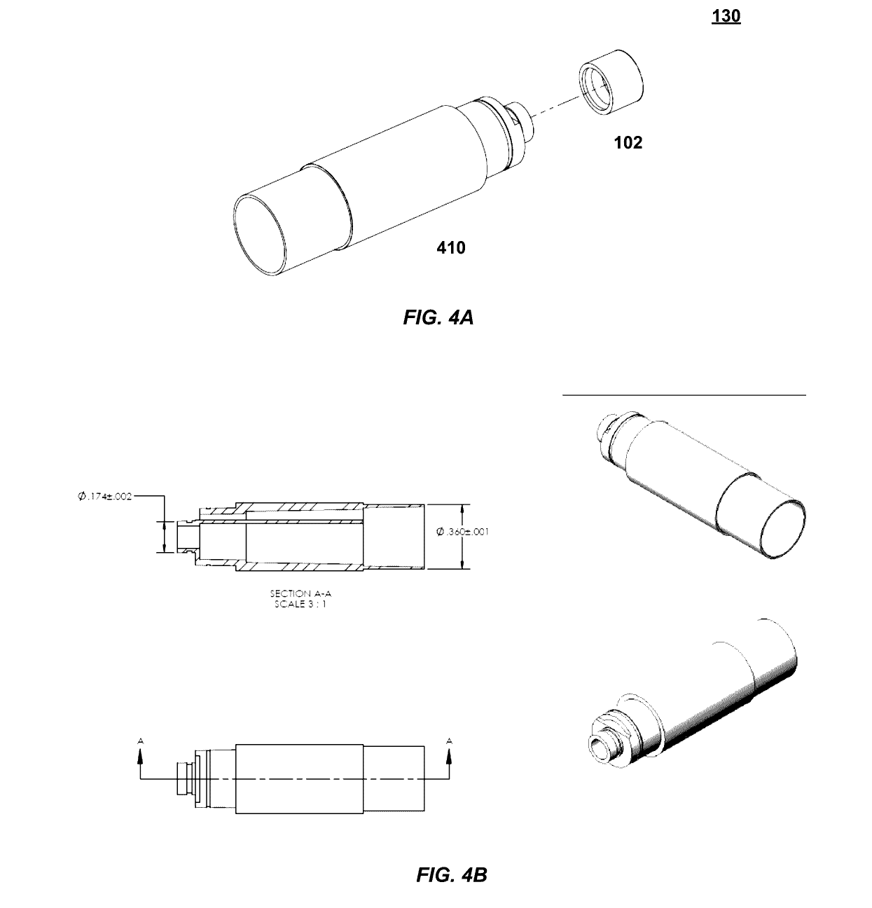

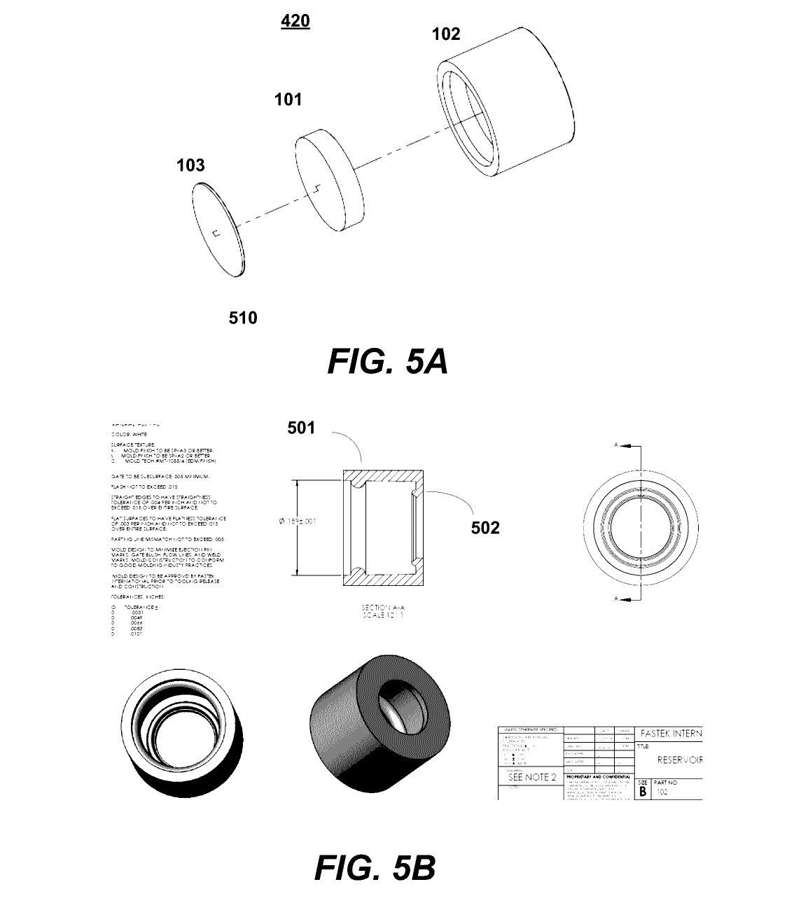

As shown in FIG. 2, the main butt assembly 110 containing a septum 101 connects to the battery tube assembly 120 by fitting a flared out end into a bored channel. The battery tube assembly 120 includes a snap tab 105, a battery tube 106, a battery 107 and a cherry 108. The main butt assembly is broken down in FIG. 3 to show a reservoir assembly 310 and an atomizer 301. A reservoir 410 of the reservoir assembly 310 is shown with a reservoir cap 420 removed in FIG. 4A. The reservoir cap 420 is shown in an exploded view in FIG. 5A to contain the septum 401 and a PFTE backing 103, both of which are affixed to the reservoir 410 when connected.

The septum 101, in this embodiment, is circular-shaped and cut from silicon or any suitable material. In more detail, the septum 101 is selected with a high durometer and coring resistance, along with and a low porosity. These characteristics allow penetration by a syringe needle 199 to fill or refill a liquid solution into the reservoir tank 410, and also resealing to prevent leaking of the liquid solution. For example, a vacuum generated by the sealed reservoir tank 410 prevents the liquid solution from seeping through holes at the bottom that are intended to aid vaporizing. The syringe needle 199 can be any appropriate type or size (e.g., 16 gauge), such as a volume consistent with a capacity of the reservoir tank 140. In one embodiment, the needle includes a hole to allow displaced air or gas to escape the reservoir tank 410 as displaced by the liquid solution. Many variations are possible with respect to septum characteristics such as, diameter, rubber type, rubber thickness, rubber color, and pre-slit or no slit, for example.

The septum 110 can be bonded on either side with a PTFE backing 103 and to the reservoir cap 520. The PFTE backing 103 can be Teflon, Silicone or any other suitable material. The PTFE backing 103 is also circular-shaped for fitting to the septum 101 and the reservoir cap 520. In combination, the anti-coring feature of the septum 101 prevents the liquid solution from leaking while the PTFE backing 103 firms up the septum 101 against pressure from the syringe needle 199 being inserted. In some implementations, the septum 101 drapes over the PTFE backing 103 to form a seal with the reservoir 410. The reservoir cap 520 can have an open area, similar to a donut hole, for access by the syringe needle 199 (the butt cap 102 has a hole for vapor inhalation that can double for syringe needle access). A size of the reservoir cap 520 is determined by a connection between the reservoir 410 and the reservoir cap 420 (e.g., a screw-threaded connection). The reservoir cap 520 size can be according to a standard (e.g., for vials) or customized. In turn, the reservoir cap size can set the boundary for sizing the septum 110 and PFTE backing 103.

Turning to a detail view of one example of the reservoir cap 520, shown in FIG. 5B, the septum 101 and PFTE backing 103 combination locks inside the reservoir cap 520 with inner diameter lips 501. The inner lips 501 can also be used to snap onto the reservoir 410 to form a seal for a liquid solution. The reservoir cap 520 also includes an inner opening 502 for syringe needle access during refills of the liquid solution.

Now turning to a detail view of the reservoir assembly 310, this time show in FIG. 4B, which holds liquid solutions intended for vaporizing. Holes (not shown) are added to some embodiments to aid vaporizing. The reservoir 410 is designed to accommodate a liquid solution, a heating element, and smoke/vapor exhaust. Conventional reservoirs are manufactured without a cap because the liquid solution is added at a later point, and then a cap can be glued or otherwise bonded permanently. Other conventional reservoirs have screw on caps that can close the reservoir after the liquid solution is added. Neither of the existing techniques addresses the improved vaporizing devices or the simplified filling and refilling processes disclosed herein.

The reservoir assembly 310 a heating coil 301 protrudes a seal 303 (e.g., a silicon sealant) in order to heat liquid solution stored in the reservoir tank 410. A compression ring 302 holds the components in place. The atomizer is powered by a battery in the battery tuber 120.

During use, a user activates the electronic vaporizer 100 by inhaling from the butt cap 104 through his or her lips. Two or more fingers can be used to stabilize the electronic vaporizer 100 when pulling vapor, similar to a cigarette when pulling smoke. The resulting vacuum force trips a sensor that turns on power so that the atomizer 301 heats up a small portion of the liquid solution (alternatively can be push-button actuated or merely powered on). Within the first few inhales, the liquid solution is converted to vapor which is pulled to an outer chamber of the reservoir tank 410. Finally, the butt cap 104 forms an outer cavity to the reservoir cap 102 for the vapor to continue through to the user's lips and inhalation into his or her lungs. The cherry 108 glows red or white to indicate when vapor is being pulled.

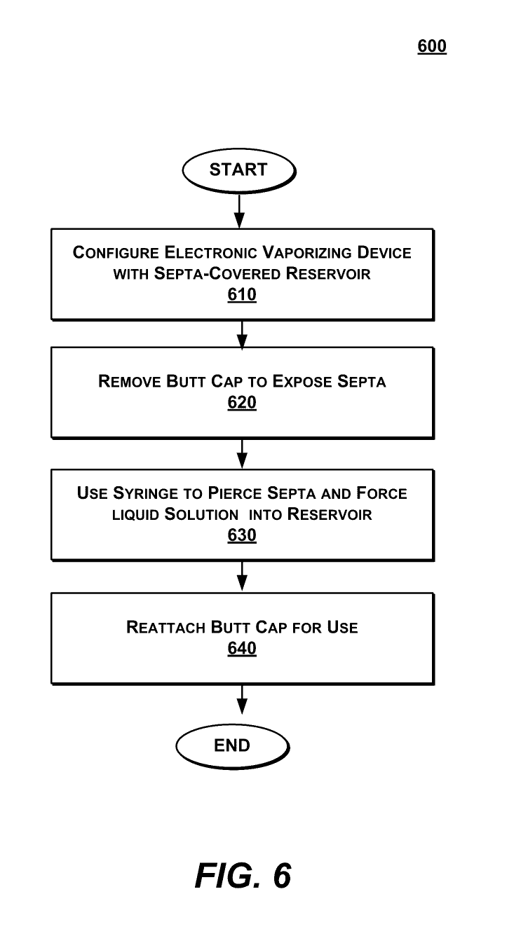

In one embodiment of a reusable electronic vaporizing device 110, the liquid solution is refillable once depleted. FIG. 6 is a flow chart illustrating a method 600 for filling a re-sealable reservoir tank with an electronic vaporizing device, according to one embodiment.

The electronic vaporizing device 110 is manufactured with the septum assembly 140 as described herein (step 610). By removing a butt cap 104, the septum 101 is exposed (step 620). Some embodiments preferably access the septum 101 from the length of the syringe needle 199 without removing the butt cap 104. Stock liquid solution can be kept in larger quantities in, for example, a glass vial 199 until transferred for refills using the syringe needle 199. A user pulls the plunger to fill up the syringe needle 199 to a volume sufficient for the reservoir cap 102 and removes the syringe needle 199 from the glass vial 198. The syringe needle 199 is then inserted through the septum 101, the plunger pushed down to force liquid solution into the reservoir 140 (step 630). The butt cap 104 is then reattached for continued use (step 640). Additional refills are possible.

In another embodiment, the electronic vaporizing device 110 is disposable and is filled with a liquid solution during manufacture. A robotic arm inserts one or an array of syringe needles into one or an array of electronic vaporizing devices at some point during assembly (e.g., through the butt cap 104 near the end of manufacturing. In either case, the reservoir 140 is filled and automatically reseals to prevent leakage from the reservoir tank 140 and reduce the timing burden of a subsequent sealing with a conventional plug. Because there will be no refills, the cap can be glued on or otherwise bonded.

In some embodiments, the electronic vaporizing device 100 is battery-powered, and in other embodiments, powered from an outlet, USB cord, or otherwise.

As will be understood by those familiar with the art, the subject matter described herein may be embodied in other specific forms without departing from the spirit or essential characteristics thereof. Likewise, the particular naming and division of the portions, modules, agents, managers, components, functions, procedures, actions, layers, features, attributes, methodologies, data structures and other aspects are not mandatory or significant, and the mechanisms that implement the subject matter or its features may have different names, divisions and/or formats. The foregoing description, for purpose of explanation, has been described with reference to specific embodiments. However, the illustrative discussions above are not intended to be exhaustive or limiting to the precise forms disclosed. Many modifications and variations are possible in view of the above teachings. The embodiments were chosen and described in order to best explain relevant principles and their practical applications, to thereby enable others skilled in the art to best utilize various embodiments with or without various modifications as may be suited to the particular use contemplated.

I claim:

1. An electronic vaporizing device having a septum membrane for filling and resealing a liquid solution, the electronic vaporizing device, comprising:
a mouth piece to inhale a vaporized form of the liquid solution;
reservoir tank sealed with the septum membrane in order to store the liquid solution, wherein the septum membrane is penetrated with a syringe needle for filling the liquid solution into the reservoir tank and, upon removal of the syringe needle, automatically resealing the reservoir tank to prevent leakage of the liquid solution;
an atomizer to vaporize a portion of the liquid solution; and
a power supply to provide power to the atomizer.

2. The electronic vaporizing device of claim 1, wherein the septum membrane is bonded with a support backing on one side and a reservoir cap on the other side, the reservoir cap including an opening for septum access by the syringe needle.

3. The electronic vaporizing device of claim 1, wherein the mouth piece covers the reservoir cap while also provides a chamber around the reservoir cap for vapor to pass from the reservoir tank to lips of the user during operation.

4. The electronic vaporizing device of claim 1, wherein the septum membrane has a durometer low enough to reseal after penetration of the syringe needle, the durometer high enough to prevent penetration of the liquid solution.

5. The electronic vaporizing device of claim 1, wherein the septum membrane has a porosity high enough for penetration of the syringe needle, the porosity low enough to prevent penetration of the liquid solution.

6. The electronic vaporizing device of claim 1, wherein the septum membrane is resistant to coring.

7. The electronic vaporizing device of claim 1, wherein the electronic vaporizing device is a disposable device.

8. The electronic vaporizing device of claim 1, wherein the electronic vaporizing device is a reusable device, and the septum membrane is re-penetrated for re-filling the reservoir tank.

9. The electronic vaporizing device of claim 1, wherein the electronic vaporizing device comprises an electronic cigarette and the reservoir tank is filled with the liquid solution containing nicotine.

10. The electronic vaporizing device of claim 1, wherein the electronic vaporizing device comprises an electronic marijuana dispenser and the reservoir tank is filled with the liquid solution containing THC.

11. The electronic vaporizing device of claim 1, wherein the electronic vaporizing device comprises an electronic marijuana dispenser and the reservoir tank is filled with the liquid solution containing a pharmaceutical or therapeutic.

12. The electronic vaporizing device of claim 1, wherein the electronic vaporizing device comprises a cigarette shape.

13. The electronic vaporizing device of claim 1, wherein the mouthpiece has a hole for both inhaling vapor and for syringe needle access to the septum.

14. The electronic vaporizing device of claim 1, wherein the reservoir tank includes permanent holes, and wherein the septum membrane generates a vacuum to prevent the liquid solution from otherwise leaking from the holes.
